(19) United States
(12) Patent Application Publication
(10) Pub. No.: US 2012/0277864 A1
(43) Pub. Date: Nov. 1, 2012

(54) CANNULA ASSEMBLY WITH NON-CIRCULAR PROFILE AND METHOD OF USE

(75) Inventors: Darrel S. Brodke, Salt Lake City, UT (US); Carl Lauryssen, Beverly Hills, CA (US); Frank M. Phillips, Highland Park, IL (US); Gary A. Schneiderman, Sacramento, CA (US); Bruce Chabansky, Palo Alto, CA (US); Calin Druma, San Jose, CA (US)

(73) Assignee: WARSAW ORTHOPEDIC, INC., Warsaw, IN (US)

(21) Appl. No.: 13/094,654

(22) Filed: Apr. 26, 2011

(51) Int. Cl. A61F 2/44

(52) U.S. Cl. 623/17.16

(57) ABSTRACT

A cannula assembly and related methods of use. The cannula assembly includes a cannula and a stylet. The cannula has an outer surface that is non-circular. Rotation of the cannula about its longitudinal axis changes the orientation of the cannula's larger outer dimension so that the cannula may be used as a distraction tool. Rotation is effected by rotating a stylet handle, which rotates the associated stylet shaft, which in turn rotates the cannula. Related methods include rotating a handle of the stylet, while the shaft of the stylet is disposed in the bore of the cannula, about the first longitudinal axis so as to rotate the cannula about the longitudinal axis to distract vertebral bodies.

### BACKGROUND

The present invention relates generally to medical devices and procedures for providing access, including percutaneous access, to a desired location in a body.

Cannulas are frequently employed to provide access to, or to deliver medical implants to, a desired location in a patient. For example, cannulas may be used to provide access to a vertebra, an intervertebral disc, or other area of the spine, to perform a variety of different medical procedures, such as percutaneous delivery of a spinal implant. Typically, such cannulas are round in cross-section, both internally and externally. While some such cannulas have tapered sections, they are not ideal for use as distracting instruments because they require longitudinal displacement toward the spine in order to provide distraction force, which can be problematic. Thus, while cannulas are widely used in spinal surgery, particularly percutaneous spinal surgeries, there remains a need for alternative designs, particularly alternative designs that are better suited for use as distraction instruments.

### Summary

The present invention provides a cannula assembly and related method(s) of use, where the cannula has an outer surface that is non-circular. Rotation of the cannula about its longitudinal axis changes the orientation of the cannula's larger outer dimension so that the cannula may be used as a distraction tool. Rotation is effected by rotating a stylet handle, which rotates the associated stylet shaft, which in turn rotates the cannula.

In some embodiments, the present invention relates to a method of inserting an implant into a space between adjacent vertebral bodies. The method may comprise inserting a guidewire to a space between adjacent vertebral bodies; thereafter, sliding a cannula assembly over the guidewire so that a distal tip of the cannula assembly extends into the space between the adjacent vertebral bodies. The cannula assembly may comprise a hollow elongate cannula and a stylet. The cannula extends along a first longitudinal axis from a proximal end to a distal end. The cannula has a generally uniform first bore therethrough with a first cross-sectional profile normal to the longitudinal axis that is non-circular. The cannula also has an outer surface having a second non-circular cross-sectional profile with a major dimension along a major axis and a minor dimension along a minor axis. The outer surface may be substantially uniform throughout the cannula's length. The stylet is sized and configured so that a portion thereof fits in the first bore of the cannula, with the stylet having a handle external to the first bore. The sliding comprises sliding the cannula assembly over the guidewire such that the minor axis of the cannula is generally parallel to the sagittal plane defined by the vertebral bodies and the major axis is generally parallel to the axial plane defined by the vertebral bodies. The method further comprises thereafter, rotating the stylet handle about the first longitudinal axis so as to rotate the cannula about the longitudinal axis to distract the vertebral bodies while the stylet is disposed in the first bore. Thereafter, the stylet is removed. The method continues with thereafter delivering a spinal implant to the space between adjacent vertebral bodies via the cannula while the cannula is oriented such that the major axis of the cannula is generally parallel to the sagittal plane defined by the vertebral bodies and the minor axis is generally parallel to the axial plane defined by the vertebral bodies. The guidewire may be removed prior to or after delivery of the implant. The cannula assembly may remain disposed over the guidewire during the rotating of the handle, which may be an approximately 90° rotation. The second cross-sectional profile associated with the cannula outer surface may comprise opposed flat sections disposed on opposing sides of the major axis and connected via curved sections that extend a generally uniform distance from the first longitudinal axis.

In some embodiments, the present invention provides a cannula assembly comprising: a hollow elongate cannula extending along a first longitudinal axis from a proximal end to a distal end. The cannula has a generally uniform first bore therethrough with a cross-sectional profile normal to the longitudinal axis that is non-circular. The cannula has an outer surface that is non-circular when viewed normal to the longitudinal axis. The cannula assembly further comprises a stylet extending into the first bore and comprising an elongate shaft and a handle. The elongate shaft of the stylet extends along a shaft axis from a proximal end to a distal end thereof. The handle is mounted to the proximal end of the stylet shaft and extends generally normal to the shaft axis. The stylet shaft has a non-circular cross-section corresponding to the cross-section of the cannula bore and substantially fills the cannula bore in cross-section. The stylet may advantageously have a tapered distal end. The stylet may further comprise a second longitudinal bore extending therethrough; the second longitudinal bore coaxial with the first bore of the cannula. The cannula outer surface has a second cross-sectional profile which may comprise opposed flat sections connected via curved sections that extend a generally uniform distance from the first longitudinal axis. The stylet is advantageously longer than the cannula, although this is not required.

In various embodiments, the present invention has one or more of the above attributes, alone or in any combination.

### BRIEF DESCRIPTION OF THE DRAWINGS

FIG. 1 shows a spinal motion segment as one exemplary location where the present invention has application.

FIG. 2 shows a cannula assembly according to one embodiment of the present invention.

FIG. 3 shows a partially exploded view of the cannula assembly of FIG. 2.

FIG. 4 shows a cross section at IV-IV of the cannula of FIG. 3.

FIG. 5 shows a cross section at V-V of the cannula assembly of FIG. 2 with the guidewire omitted for clarity.

FIG. 6 shows a guidewire extending to the disc space through the Kambin triangle.

FIG. 7 shows a cannula assembly being slid over the guidewire of FIG. 6.

FIG. 8 shows the cannula assembly of FIG. 6 after rotation.

FIG. 9 shows insertion of a spinal implant into the cannula of FIG. 8.

### DETAILED DESCRIPTION

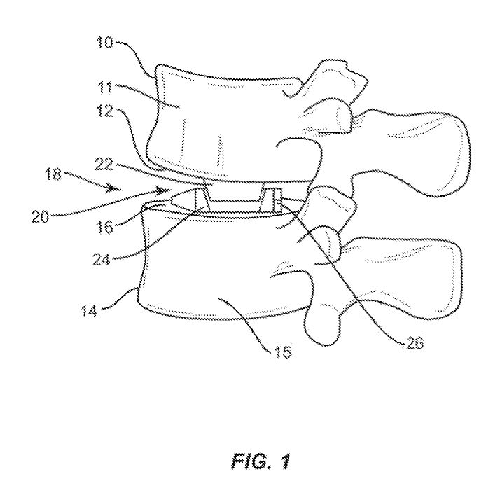

In one embodiment, the present invention is directed to a cannula assembly 40 and/or method of using a cannula assembly 40 during spinal surgery. In order to provide illustrative context, the following discussion will focus primarily on use of the invention for spinal surgery in the lumbar region of the spine, but it should be understood that the invention may alternatively or additionally be used in other regions of the spine. FIG. 1 depicts adjacent vertebrae 10,14 of the lumbar region of a human spinal column. Each vertebrae 10,14 comprises a corresponding vertebral body 11,15, a superior articular process, a transverse process, an inferior articular process, and a spinous process. In addition, between vertebral bodies 11,15 is a space 18 normally occupied by an intervertebral disc and bounded by the endplates 12, 16 of the vertebral bodies. Due to various conditions, such as a collapsed disc, it may be desired to place an implant in the disc space 18 in order to provide proper structural continuity between the vertebral bodies 11,15. The cannula assembly 40 of the present invention provides a convenient means for delivery of the implant to the desired location.

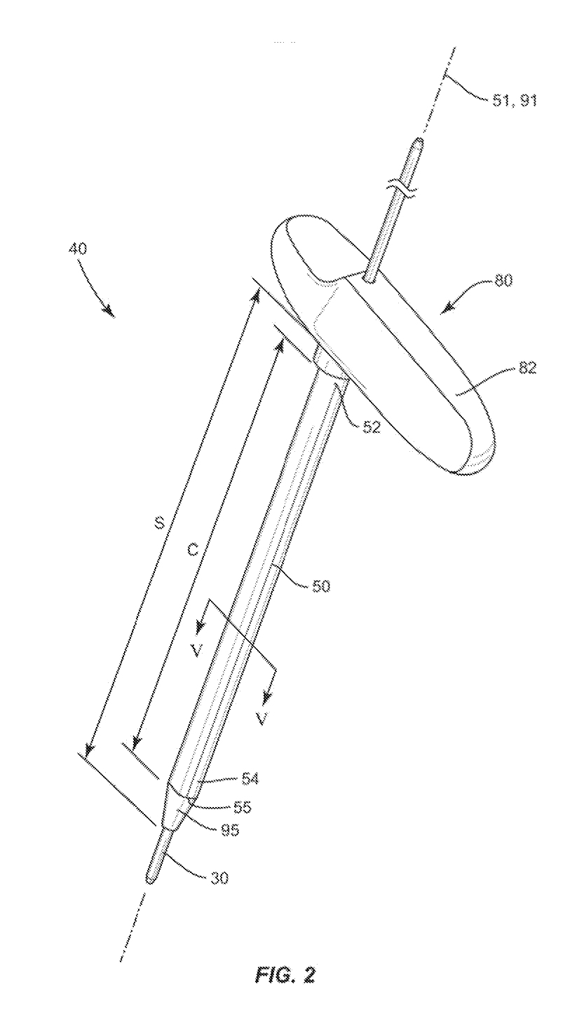

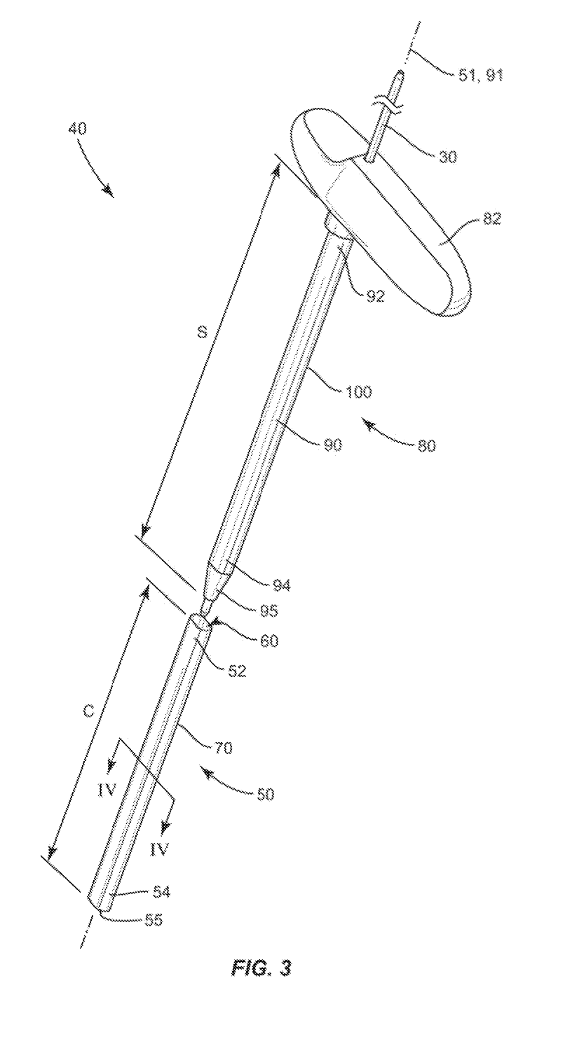

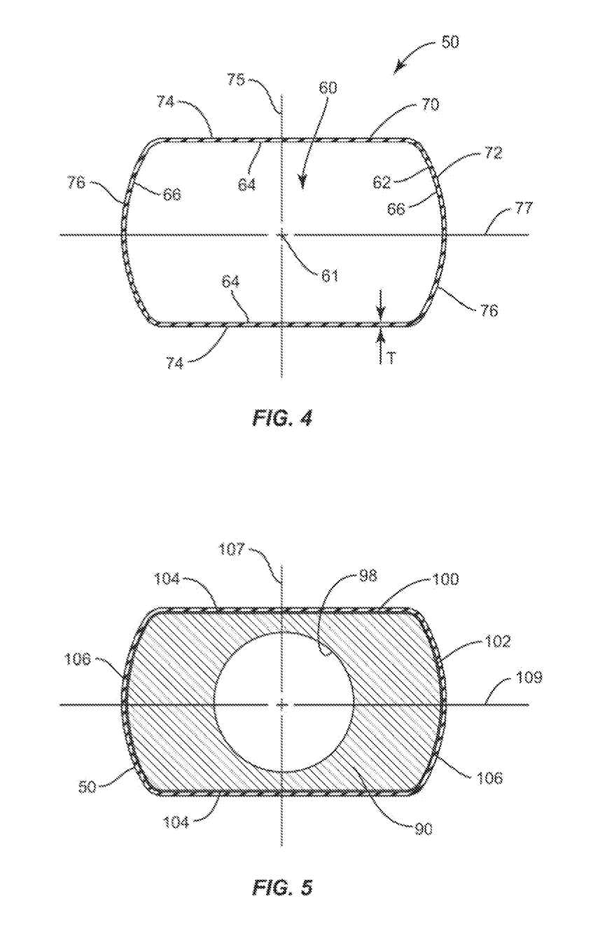

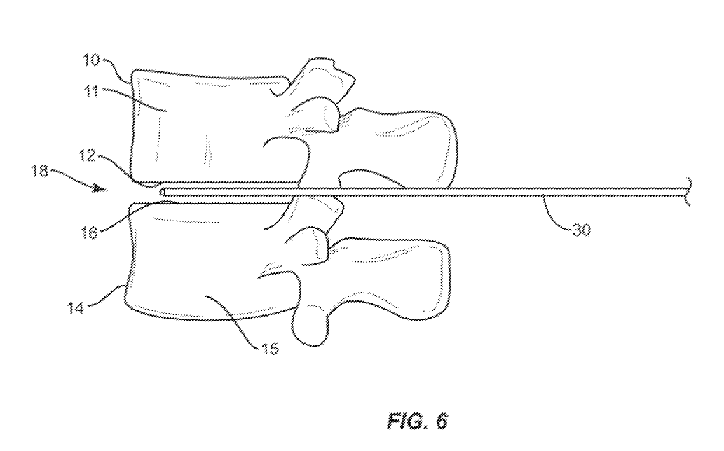

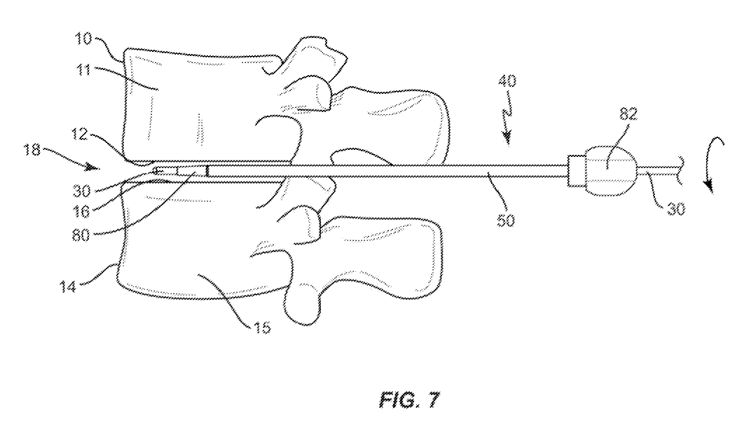

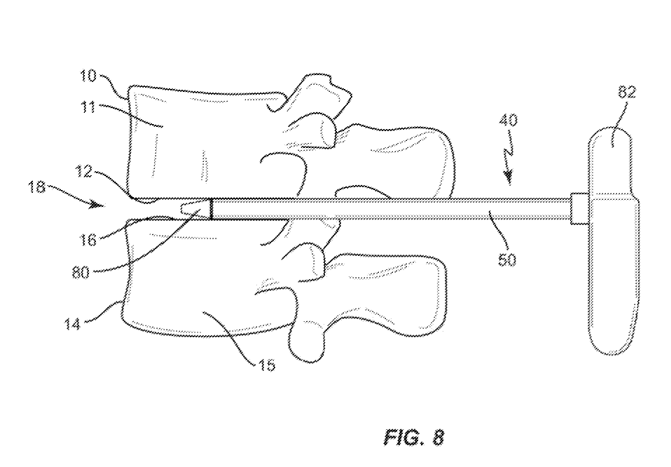

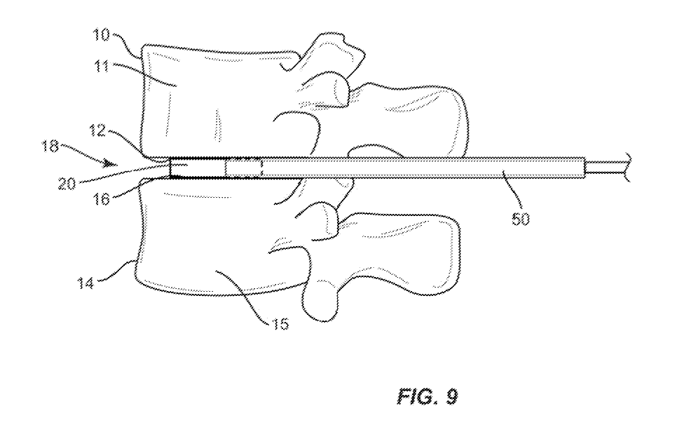

The cannula assembly 40 of FIGS. 2-3 includes a cannula 50 and a stylet 80. The cannula 50 is a hollow elongated body that extends along a longitudinal axis 51 from a proximal end section 52 to a distal end section 54. A distal edge 55 lies at the distal most portion of the distal end section 54. The overall longitudinal length of the cannula 50 is C. A bore 60 extends entirely through the cannula 50 and is advantageously concentric with the exterior surface 70 and substantially uniform throughout its length. The bore 60 is non-circular in cross section. That is, the perimeter 62 of the bore 60 has a profile, normal to axis 51, that is non-circular. Advantageously, the perimeter profile is partially round, with two flat sections 64 disposed opposite each other and connected by two curved sections 66. The curved sections 66 advantageously are portions of a circle centered on the bore's axis 61, which is collinear with the axis 51 of cannula 50. Likewise, the outer surface 70 of the cannula 50 is non-circular in cross section, advantageously along its entire length, but at least through the entire distal end section 54. That is, the profile of the cannula outer surface perimeter 72, normal to axis 51, is non-circular. Advantageously, the profile is partially round, with two flat sections 74 disposed opposite each other and connected by two curved sections 76. Due to the flats 74, the major axis 77 of the cannula cross section extends through the curved sections 76 and parallel to the flats 74, while the minor axis 75 of the cannula cross section extends through the flat sections 74 and perpendicular to the major axis 77. The curved sections 76 advantageously are portions of a circle centered on the bore's axis 61. Thus, the cannula 50 advantageously has a generally constant wall thickness T, although such is not required in all embodiments.

The stylet 80 includes a handle 82 and an elongated shaft 90. The shaft 90 is designed to slide in the bore 60 of the cannula 50, and extends along a shaft axis 91 from proximal section 92 to a distal section 94. The distal section 94 advantageously includes a tip section 95 that tapers in the distal direction. The length of the shaft is S, which is longer than the length C of the cannula 50. The shaft 90 advantageously includes a shaft bore 98 extending entirely therethrough. The outer surface 100 of the shaft 90 is non-circular and corresponds to the shape of the cannula bore 60. Thus, the shaft outer surface 100 advantageously has a cross sectional profile with two flat sections 104 on opposing sides and connected by curved sections 106. The minor axis 107 of the shaft extends through flat sections 104, while the major axis 109 of the shaft extends through the curved sections 106. When disposed in the cannula 50, the shaft 90 advantageously substantially fills the cannula bore 60, such that the outer surface 100 of the shaft 90 and the inner surface of the cannula bore 60 are for practical purposes in contact along the entire perimeter 62 and along the entire length of the bore 60 with a sliding fit. The handle 82 is mounted to the proximal section 92 of the shaft 90, and extends generally perpendicular to the shaft axis 91. The handle 82 may extend in one or advantageously both directions away from the shaft 90. Any suitable method may be used to join the handle 82 to the shaft 90, such as by adhesives, including suitable ears on the shaft 90 that are captured by the handle 82, spline connections, or the like. If desired, a portion of the proximal section 92 of shaft 90 may be enlarged for better mating with handle 82. Further, if desired, a shoulder may be formed at the edge of such enlargement so that a positive stop is formed to prevent over-insertion of the stylet 80 into the cannula 50.

The components of the cannula assembly 40 may be made from any suitable materials known in the art. Just by way of example, the cannula 50 and stylet shaft 90 may be made from stainless steel, while the handle 82 may be made from suitable plastics known in the art.

As mentioned above, the cannula assembly 40 may be employed to help install a spinal implant 20. One such spinal implant 20 includes a first vertebra engaging portion 22 moveably coupled to a second vertebra engaging portion 24. Movement of an actuator 26 causes the implant 20 to move from a collapsed configuration to an expanded configuration, by moving the first and second vertebra engaging portions 24,26 away from each other so as to increase the effective height of the implant 20. For additional information about such type of implants, see U.S. Patent Application Publication Nos. 2009/0198337 and 2006/0206207, and/or U.S. Pat. No. 7,217,293. Of course, other implants 20 may be delivered via the cannula assembly 40, the particular details of which are unimportant to understanding the present invention. Indeed, while expandable implants 20 are believed advantageous, non-expandable implants 20 may alternatively or additionally be employed.

The cannula assembly 40 may be used to help position a spinal implant 20 in the disc space 18 between the adjacent vertebrae 10,14. One illustrative method of doing so includes preparing the surgical site in a conventional fashion, and then inserting a guidewire 30 into the disc space 18, with the outer end of guidewire 30 being outside the patient's body. A posterior-lateral approach may be used. The guidewire 30 is advantageously routed to the disc space 18 via the Kambin triangle defined by the corresponding traversing and exiting nerves. The cannula assembly 40 is slid over the guidewire 30 by running the guidewire 30 through the bore 98 of the stylet 80 and moving the cannula assembly 40 toward the disc space 18. The stylet 80 extends distally from the cannula 50, so the tapered tip 95 of the stylet 80 leads the cannula assembly 40. The transition from the stylet 80 to the cannula 50 is minimized by the tapered edge 55 of the cannula 50, which may be similar to that described in U.S. Patent Application Publication No. 2007/0260184. During this sliding on the guidewire 30, the cannula 50 is oriented so that flats 74 on the cannula outer surface 70 are parallel to the planes endplates 12,16 of the vertebral bodies 11,15. Thus, the cannula 50 is inserted into the disc space 18 in its “vertically thinner” orientation. The surgeon then grips the handle 82 and rotates the stylet 80 about the shaft axis 91. Because the stylet 80 and the cannula bore 60 are non-circular, this causes the cannula 50 to rotate about its axis 51. Preferably, the rotation is approximately, or exactly, 90° so that the midpoints of the curved sections 76 of the cannula outer surface 70 press against the endplates 12,16. Because the curved sections 76 lie along the major axis 77, and are therefore farther apart than the flat sections 74, the vertebral bodies 11,15 are distracted apart due to the rotation of the cannula 50. Thus, the cannula 50 is inserted between the vertebral bodies 11,15 oriented so that the minor axis 75 is parallel to the sagittal plane, and rotated so that the major axis 77 is parallel to the sagittal plane, thereby increasing its effective height for separating the vertebral bodies 11,15.

The stylet 80, and optionally the guidewire 30, are then removed, leaving the cannula 50 in place. The implant 20 is then inserted into the proximal end 52 of the cannula 50, moved along the cannula bore 60, and pushed out the distal end 54 of the cannula 50 into the disc space 18. A push rod may be used for this displacement of the implant 20, and/or a suitable actuation instrument may be used. An actuator instrument may then be used, if appropriate, to further increase the height of the implant 20 by moving its first and second vertebral engaging portions 22,24 apart. The cannula 50 is then removed, and the surgical procedure continues as is conventional. If desired, multiple cannula assemblies 40 may be used to install corresponding multiple implants 20 into the same disc space 18, and/or into multiple disc spaces 18.

The discussions above have been in the context of using a guidewire 30 to guide the cannula assembly 40 into place. It should be understood that a “guidewire” may be a flexible or semi-flexible wire (metallic or otherwise), or a more rigid body like a rod. Further, while use of a guidewire 30 is advantageous, such is not strictly required for all embodiments. Further, the guidewire 30 may be removed either prior to or after rotation of the cannula 50, as is desired. And, the discussion has assumed that the cannula assembly 40 is slid over the guidewire 30 as a unit. However, such is not required in all embodiments, and the stylet 80 and the cannula 50 may be slid over the guidewire 30, or otherwise positioned prior to rotation, in any sequence or simultaneously.

The cannula assembly 40 may alternatively be inserted with the major axis 77 of the cannula 50 parallel with the sagittal plane. For such a situation, distraction may be achieved by the tapered tip section 95 of stylet 80, rather than by rotation of the cannula 50.

All U.S. patents and patent application publications mentioned above are hereby incorporated herein by reference in their entirety.

The present invention may, of course, be carried out in other specific ways than those herein set forth without departing from the scope of the invention. The present embodiments are, therefore, to be considered in all respects as illustrative and not restrictive, and all changes coming within the meaning and equivalency range of the appended claims are intended to be embraced therein.

What is claimed is:

1. A method of inserting an implant into a space between adjacent vertebral bodies, comprising:
inserting a guidewire to a space between adjacent vertebral bodies;
thereafter, sliding a cannula assembly over the guidewire so that a distal tip of the cannula assembly extends into the space between the adjacent vertebral bodies;
the cannula assembly comprising:
a hollow elongate cannula extending along a first longitudinal axis from a proximal end to a distal end; the cannula having a generally uniform first bore therethrough with a first cross-sectional profile normal to the longitudinal axis that is non-circular;
the cannula having an outer surface having a second non-circular cross-sectional profile with a major dimension along a major axis and a minor dimension along a minor axis;
a stylet disposed in the first bore and having a handle external to the first bore;
wherein the sliding comprises sliding the cannula assembly over the guidewire such that the minor axis of the cannula is generally parallel to the sagittal plane defined by the vertebral bodies and the major axis is generally parallel to the axial plane defined by the vertebral bodies;
thereafter, rotating the handle of the stylet about the first longitudinal axis so as to rotate the cannula about the longitudinal axis to distract the vertebral bodies while the stylet is disposed in the first bore;
thereafter, removing the stylet;
thereafter, delivering a spinal implant to the space between adjacent vertebral bodies via the cannula while the cannula is oriented such that the major axis of the cannula is generally parallel to the sagittal plane defined by the vertebral bodies and the minor axis is generally parallel to the axial plane defined by the vertebral bodies.

2. The method of claim 1 further comprising removing the guidewire prior to the delivering the spinal implant.

3. The method of claim 1 wherein the second cross-sectional profile comprises opposed flat sections disposed on opposing sides of the major axis and connected via curved sections that extend a generally uniform distance from the first longitudinal axis.

4. The method of claim 1 wherein the first cross sectional profile is a reduced scale version of the second cross sectional profile.

5. The method of claim 1 wherein the inserting the guidewire comprises inserting the guidewire through the Kambin triangle of the associated spinal segment.

6. The method of claim 1 wherein the cannula assembly remains disposed over the guidewire during the rotating of the handle.

7. The method of claim 1 wherein the rotating of the handle comprises rotating the handle 90° about the first longitudinal axis.

8. The method of claim 1 wherein the shaft of the stylet is metallic and rigid.

9. The method of claim 1 further comprising thereafter expanding the spinal implant.

10. The method of claim 1 wherein the cannula comprises a tapered edge on its distal end.

11. The method of claim 1:
wherein the stylet comprise an elongate shaft and the handle; the elongate shaft extending along a shaft axis from a proximal end to a distal end thereof; the handle mounted to the proximal end and extending generally normal to the shaft axis;
wherein the stylet shaft has a non-circular cross-section corresponding to the cross-section of the cannula bore and substantially filling the cannula first bore in cross-section when the stylet is fully inserted into the first bore;
wherein the stylet is disposed in the bore during the sliding of the cannula assembly over the guidewire.

12. The method of claim 1 wherein the sliding the cannula assembly over the guidewire comprises sliding the cannula and the stylet over the guidewire simultaneously.

13. The method of claim 1:
wherein the second cross-sectional profile comprises opposed flat sections disposed on opposing sides of the major axis and connected via curved sections that extend a generally uniform distance from the first longitudinal axis;
wherein the first cross sectional profile associated with the cannula bore is a reduced scale version of the second cross sectional profile;
wherein the stylet comprise a rigid elongate shaft and the handle; the shaft extending along a shaft axis from a proximal end to a distal end thereof; the handle mounted to the proximal end and extending generally normal to the shaft axis;
wherein the stylet shaft has a non-circular cross-section corresponding to the cross-section of the cannula bore and substantially filling the cannula first bore in cross-section when the stylet is fully inserted into the first bore;
wherein the rotating of the handle comprises rotating the handle 90° about the first longitudinal axis.

14. The method of claim 13:
wherein the inserting the guidewire comprises inserting the guidewire through the Kambin triangle of the associated spinal segment;
wherein the sliding the cannula assembly over the guidewire comprises sliding the cannula and the stylet over the guidewire simultaneously;
further comprising removing the guidewire prior to the delivering the spinal implant.

15. A cannula assembly comprising:
a hollow elongate cannula extending along a first longitudinal axis from a proximal end to a distal end;
the cannula having a generally uniform first bore therethrough with an inner cross-sectional profile normal to the longitudinal axis that is non-circular; the cannula having an outer surface that is non-circular when viewed normal to the longitudinal axis;
a stylet extending into the first bore and comprising an elongate shaft and a handle;
the elongate shaft extending along a shaft axis from a proximal end to a distal end thereof; the handle mounted to the proximal end and extending generally normal to the shaft axis;
the stylet shaft having a non-circular cross-section corresponding to the cross-section of the cannula bore and substantially filling the cannula bore in cross-section.

16. The cannula assembly of claim 15 wherein the stylet has a tapered distal end.

17. The cannula assembly of claim 15 wherein the stylet further comprises a second longitudinal bore extending therethrough; the second longitudinal bore coaxial with the first bore of the cannula.

18. The cannula assembly of claim 15 wherein the cannula outer surface has a second cross-sectional profile comprising opposed flat sections connected via curved sections that extend a generally uniform distance from the first longitudinal axis.

19. The cannula assembly of claim 15 wherein the stylet proximal end is disposed proximal to the cannula proximal end and wherein the stylet distal end is disposed distal to the cannula distal end.
